(19) United States
(12) Patent Application Publication
(10) Pub. No.: US 2026/0086100 A1
(43) Pub. Date: Mar. 26, 2026

(54) Method of Determining Risk for Chronic Stress and Stroke

(71) Applicant: NORTH-WEST UNIVERSITY, Potchefstroom (ZA)

(72) Inventors: Leoné MALAN, Potchefstroom (ZA); Nicolaas Theodor MALAN, Potchefstroom (ZA)

(21) Appl. No.: 19/406,499

(22) Filed: Dec. 2, 2025

Related U.S. Application Data
(63) Continuation-in-part of application No. 17/632,078.

(30) Foreign Application Priority Data
Aug. 1, 2019 (ZA) 2019/05103

(51) Int. Cl. G01N 33/68; A61B 5/16; A61B 5/361; G01N 33/573; G01N 33/70; G01N 33/72; G01N 33/74; G01N 33/92; G01N 33/94; G16H 50/30

(52) U.S. Cl. CPC G01N 33/6893; A61B 5/165; A61B 5/361; G01N 33/573; G01N 33/6896; G01N 33/70; G01N 33/723; G01N 33/74; G01N 33/743; G01N 33/92; G01N 33/94; G16H 50/30; G01N 2800/2871; G01N 2800/50; G01N 2800/7004

(57) ABSTRACT

Provided are methods of determining risk for chronic stress and stroke. More specifically, provided is an early prognostic index that can be used to predict chronic stress and stroke risk. There is provided a method of evaluating the risk of developing chronic stress and stroke, the method including obtaining a biological sample from an individual; measuring the levels of a set of biomarkers in the biological sample obtained from the individual; measuring the levels of a set of clinical markers of the individual; using a computer to programmatically generate an index based on the levels of biomarker in the biological sample obtained from the individual in combination with levels of the individual's clinical marker; and using the index to identify a likelihood that the individual will experience chronic stress and stroke.

### CROSS-REFERENCE TO RELATED APPLICATION

This present application is a continuation-in-part application of U.S. application Ser. No. 17/632,078, filed Feb. 1, 2022, which is a National Stage Application of International Application Number PCT/IB2020/057269, filed Jul. 31, 2020, which claims priority to South Africa Patent Application No. 2019/05103, filed Aug. 1, 2019, the disclosure of which is hereby incorporated by reference in its entirety, including all figures, tables, and drawings.

### BACKGROUND

The present invention relates to methods of determining risk for chronic stress and stroke. More specifically, the invention relates to an early prognostic index that can be used to predict chronic stress and stroke risk.

Despite significant advances in medical technology and treatment programs, cardiovascular disease (CVD) and stroke remain the leading cause of death for both men and women worldwide. The American Heart Association lists seven key health and behavioral factors that increase risk for heart disease and stroke. This list does not include chronic emotional stress (hereafter stress), despite the fact that the World Health Organization (WHO) regards stress as one of the leading causes of disability worldwide. An individual that experiences increased levels of stress could therefore also experience an increased risk of developing stroke.

The effective management of stress could be beneficial to the prevention and therapy of ischemic heart disease and stroke and could be of major public health importance. Furthermore, individuals can be screened for elevated levels of several risk factors that could contribute to chronic stress and stroke. For example, elevated low-density-lipoprotein cholesterol (LDL) levels have been shown to increase the risk of developing stroke. The protein Troponin T (Trop T) may also be indicative of chronic stress, where increased levels of Trop T can be associated with tissue damage in heart muscle and may support the differential diagnosis of coronary versus non-coronary heart diseases.

Whilst stress has often been excluded as a risk factor for ischemic heart disease and stroke, accumulating data would suggest that stress may trigger perfusion deficits leading to ischemic heart disease and stroke risk. The overlap between symptoms of perfusion deficits and stress, such as palpitations, chest pain, and shortness of breath regularly occurring in healthy persons at emergency departments, makes it difficult to utilize mental health status as a diagnostic tool in ischemic heart disease. However, perfusion deficit symptoms are regularly treated and risk factors are evaluated without addressing emotions as a possible contributing factor in the condition. Moreover, the hesitancy of patients to discuss mental health issues may also increase the risk of stroke.

Presently, screening tests and preventative measures for chronic stress and stroke are limited. An index or tool that utilizes statistical analyses of various risk factors or markers of each individual might therefore prove to be useful as a predictor of chronic stress and stroke risk. The index or tool might further provide an early prognostic index in healthy or high-risk individuals in preventive medicine via phenotyping. Phenotyping explains how genetic and environmental influences come together to create an organism's physical appearance and behavior. Such a phenotype can be determined by measuring risk factors that can be analysed from a sample that has been obtained from an individual. Other risk factors may include, but are not limited to, age, gender, systolic blood pressure, hypertensive drugs, diabetes and smoking habit. The various risk factors can then be individually weighted and contribute to a final index or tool, which can be capable of determining or predicting whether an individual has a high risk of developing chronic stress and stroke.

### BRIEF SUMMARY

Accordingly, it is an object of the present invention to provide a stress screening tool and with which the applicant believes the aforementioned disadvantages may at least partially be alleviated or which may provide a useful alternative for the known systems and methods.

According to a first aspect of the invention, there is provided a method of evaluating the risk of developing chronic stress and stroke, the method including:
obtaining a biological sample from an individual;
measuring levels of one or more biomarkers in the biological sample obtained from the individual;
measuring levels of one or more clinical markers of the individual;
using a computer to programmatically generate an index based on the levels of the one or more biomarkers in the biological sample obtained from the individual in combination with the levels of the one or more of the individual's clinical markers; and
using the index to identify a likelihood that the individual will experience chronic stress and stroke.

In an embodiment of the invention, the individual may be a human.

The biological sample obtained from the individual may be selected from the group consisting of blood, serum, plasma, urine, saliva and a combination of one or more thereof.

In an embodiment of the invention, the one or more biomarkers may be selected from the group consisting of adrenocorticotrophic hormone (ACTH), cortisol, catecholamines (norepinephrine, epinephrine), low-density lipoprotein (LDL), high-density lipoprotein (HDL), troponin T (Trop T), gamma-glutamyl transferase (γ-GT), glycated hemoglobin (HbA_{1C}), high sensitivity C-reactive protein (CRP), cotinine and a combination of one or more thereof. Furthermore, the one or more biomarkers may be determined by a method of either immunoassay or enzymatic activity assay.

In terms of the invention, the one or more clinical markers may be selected from the group consisting of age, race, gender, physical activity, medical history, smoking habits, alcohol habits, systolic blood pressure, diastolic blood pressure, perfusion deficits (24 h myocardial ischemia events), electrocardiography (ECG) atrial fibrillation, left ventricular hypertrophy (ECG-LVH) and a combination of one or more thereof.

The calculation of the index may be performed using a suitably programmed computer. Furthermore, the index may be a risk score or an equivalent thereof.

Calculation of the index may include the steps of:
logarithmically transforming the measured levels of one or more biomarkers and one or more clinical markers to generate transformed data;
multivariate regression model using transformed data to predict stroke risk for all data, 10 training sets and 10 test sets;
logistic regression using continuous predictors to predict stroke risk in all data and 10 training sets, maximum likelihood estimates (p-value);
receiver operating characteristics area under the curve (AUC) in all data and 10 training sets predicting stroke risk;
pearson correlation coefficients for logits of predicted probabilities, based on the stroke risk cut point, with transformed risk components in all data, 10 training and 10 test sets;
logistic regression using dichotomous predictors to predict stroke risk, presenting maximum likelihood estimates (p-value) for all data and 10 training sets;
receiver operating characteristics area under the curve (AUC) in all data and 10 training sets predicting stroke risk with dichotomous predictors. Hosmer-Lemeshow tests were performed to test the goodness of fit for the logistic regression risk prediction models (in all participants and 10 training sets);
logistic regression using dichotomous predictors at baseline and 3-year follow-up to predict stroke risk, presenting maximum likelihood estimates (p-value) for all data; and
receiver operating characteristics area under the curve (AUC) at baseline and 3-year-follow-up predicting stroke risk with dichotomous predictors.

In an embodiment of the invention, the method may include identifying the individual as having an increased likelihood of having a chronic stress and stroke related condition if the generated index is greater than a reference index, and identifying the individual as having a decreased likelihood of having a chronic stress and stroke condition if the generated index is less than the reference index. The reference index may be a standard or a threshold.

The invention may include recommending or authorizing treatment by authorized medical personnel if the individual is identified as having an increased likelihood of the chronic stress and stroke condition.

According to a second aspect of the invention, there is provided a method for evaluating the risk of developing chronic stress and stroke in an individual using a computer readable medium having computer executable instructions in a smartphone, the method including:
inputting into a computer the levels of one or more biomarkers measured in the biological sample obtained from the individual;
inputting into a computer the levels of one or more clinical markers of the individual;
retrieving a programmatically generated index based on the levels of one or more biomarkers and one or more clinical markers from the computer; and
displaying the index on a screen of the computer.

In an embodiment of the invention, there is provided outputting the index to a user interface device, a local computer system or a remote computer system, or a computer readable storage medium.

In an embodiment of the invention, there is provided transmitting, storing, displaying or printing the information related to the likelihood of chronic stress and stroke in an individual.

According to a third aspect thereof, the invention provides for use of the method according to the first and/or second aspect of the invention for detecting, identifying, predicting, improving the prediction accuracy of and/or facilitating a therapeutic decision for chronic stress and stroke condition in an individual.

These and other aspects of the present invention will now be described in more detail herein and below.

### BRIEF DESCRIPTION OF DRAWINGS

The invention will now be described further, by way of example only, with reference to accompanying figures.

FIG. 1 shows a design of the bi-ethnic sex cohort of the Sympathetic Activity and Ambulatory Blood Pressure (SABPA) in Africans prospective study.

FIG. 2 shows linear regression analyses receiver operating characteristic (ROC) curve depicting chronic stress in the prediction of UCLA 10-year stroke risk score (STRESS^{risk}). The area under the curve, AUC (95% CI) was 0.77 (95% CI 0.72, 0.82) for a positive prediction (85% sensitivity; 48% specificity at a cut point of 0.25) in randomly selected samples (10 training sets, each 60% of population) and 10 test sets (the remaining 40%); and

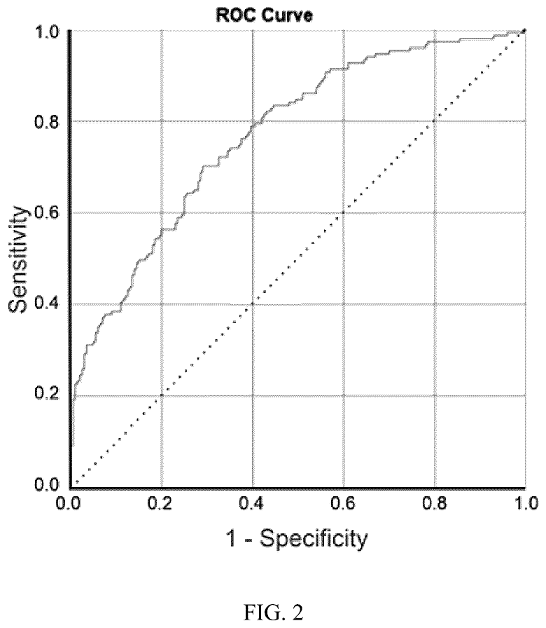

FIG. 3 shows non-linear regression analyses receiver operating characteristic (ROC) curves determined Youden indexes for the probability of chronic stress to predict a UCLA 10-year stroke risk score in the same 10 randomly selected samples (training sets, each 60% of population) and 10 test sets (the remaining 40%) as used in the linear logistic regression analyses.

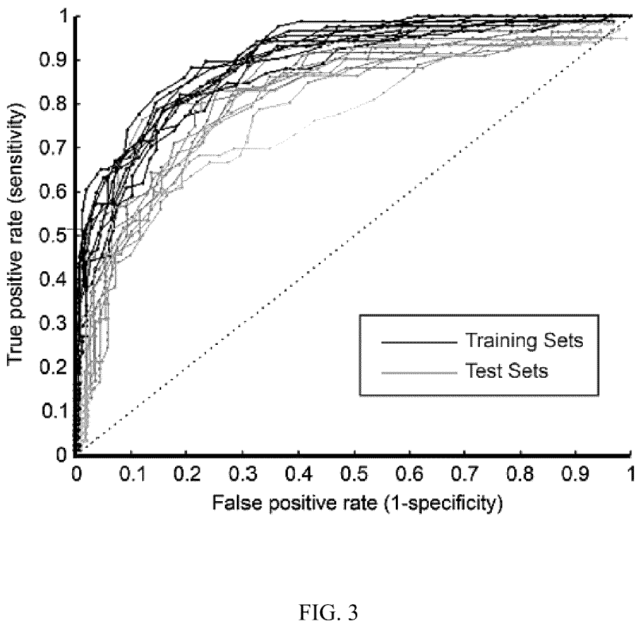

FIG. 4 shows design of the Sympathetic Activity and Ambulatory Blood Pressure (SABPA) in Africans prospective study.

FIG. 5 shows a receiver operating characteristic (ROC) curve depicting a STRESS^{d-risk} index cut point of 48.43 in the prediction of the probability of the original UCLA 10-year stroke risk. The area under the curve (AUC) (95% CI) was 0.78 (95% CI 0.73; 0.83) for a positive prediction with 81% sensitivity/59% specificity.

FIG. 6 shows a receiver operating characteristic (ROC) curve of selected input continuous stress biomarkers (V) with dependent variable (Y); a novel 10-year stroke risk marker. A cut point of 46.1% depicted the predicted probability of positives (V). The area under the curve (AUC) (95% CI) was 0.82 (95% CI 0.75; 0.85); p≤0.001 for a positive prediction with 85% sensitivity/58% specificity.

FIG. 7 shows a table of probability of stroke within 10 years for men 55-84 years and free of previous stroke (see also D'Agostino et al., Stroke risk profile: Adjustment for antihypertensive medication, The Framingham study, Stroke, 1994, 25:40-43, doi: 10.1161/01.str.25.1.40).

FIG. 8 shows a table of probability of stroke within 10 years for women 55-84 years and free of previous stroke (see also D'Agostino et al., supra.).

### DETAILED DESCRIPTION

The invention described herein is not to be limited in scope by the specific embodiments herein disclosed, as the embodiments are intended as illustrative of several aspects of the invention. Any equivalent embodiments are intended to be within the scope of this invention, as they will become apparent to those skilled in the art from the present description.

The present invention provides a method of determining the risk of chronic stress and stroke in an individual. Accordingly, biomarkers and clinical markers can be useful in assessing the health state or status of an individual by using a weighted analysis of the levels of one or more biomarkers and one or more clinical markers to generate an index for an individual.

The term “ischemic heart disease” also called coronary heart disease or coronary artery disease has previously been described in the Sympathetic Activity and Ambulatory Blood Pressure in Africans (SABPA) study (Malan, et al., 2017). Briefly, ischemia is defined by inadequate blood supply due to narrowing of blood vessels that supply blood and oxygen to the heart muscles. While various factors contribute to the narrowing or constriction of the blood vessels, the interruption in blood supply ultimately results in cellular death of the heart muscles, which lead to complications of the heart during exercise or emotional stress, where an increase in demand of oxygen is experienced, but not adequately met.

The term “stroke” is described as an interruption in the blood supply to the brain, majority (85%) of which is the result of ischemia. Similar to ischemic heart disease, the interruption of blood supply occurs due to occlusions in micro blood vessels, but which affects the brain (including retinal vessels) instead of the heart. While diabetes is a separate condition from heart disease, it shares similar threads in that they both affect blood vessels and risk for stroke. Diabetes is a clinical condition present when there is abnormal glucose regulation—where chronically raised levels of glucose is known as hyperglycemia. Moreover, as diabetes is a known independent risk factor for stroke, both heart disease and diabetes share similar characteristics when it comes to management of the diseases.

In an embodiment, a method for determining a stress-risk index for the risk of developing chronic stress and ischemic heart disease related stroke can comprise:
i) performing a multiple stepwise linear regression of biomarkers and risk factors, both transformed to be normally distributed, of an adapted UCLA score, wherein said adapted UCLA score is a 10-year stroke risk composite score of the University of California, Los Angeles (UCLA), and wherein the adapted UCLA score includes as variables: individual's medical history regarding any cardiovascular disease, kidney disease, myocardial infarction, diabetes, and hypertension medication usage; demographic and lifestyle factors including age, race, sex, diabetes, smoking, alcohol use, and physical activity habits; systolic and diastolic blood pressure; fibrinogen; waist circumference; perfusion deficits including myocardial ischemia; electrocardiography atrial fibrillation; and electrocardiography left ventricular hypertrophy; wherein the biomarkers and risk factors have been determined by obtaining biological samples from individuals; measuring levels of the biomarkers in the biological samples obtained from the individuals and measuring levels of clinical markers of the individuals, wherein the biomarkers are serum cotinine values, gamma glutamyl transferase (γ-GT), lipids and high sensitivity c-reactive protein (CRP), whole blood EDTA glycated hemoglobin (HbA1C), citrate fibrinogen values, saliva cortisol, serum cortisol, adrenocorticotrophic hormone (ACTH), high sensitivity cardiac troponin, urinary norepinephrine, epinephrine, and creatinine, wherein the clinical markers are systolic blood pressure (SBP), diastolic blood pressure (DBP), silent myocardial ischemia (SMI) events or perfusion deficits, electrocardiography (ECG) atrial fibrillation, ECG left ventricular hypertrophy (ECG-LVH), retinal vessel calibers, intra-ocular pressure (IOP), and diastolic ocular perfusion pressure (DOPP) calculated from DBP minus IOP;
ii) performing a receiver operating characteristic (ROC) analysis to assess a difference between the distribution of biomarkers and the distribution of risk factors at all classification thresholds;
iii) determining a stress-risk index for three or more possible combinations of the biomarkers as an area under the curve (AUC) as a maximum of the ROC analysis, when discriminating positives and negatives of a composite dichotomous biomarker of the adapted UCLA score denoted as Y, wherein the stress-risk index AUC was 0.77 (with a 95% confidence interval (CI) of 0.72, 0.82), for a positive prediction (with 85% sensitivity, and 48% specificity);
iv) determining a stress-risk index cut-off value by a Youden index that maximizes correct classifications and/or minimizes incorrect classifications and denotes a combination of biomarkers at a determined optimal cut-off value as biomarker V;
v) validating biomarker V by using Y as a dependent variable and V as a predicted probability of positives using a logistic regression model on input continuous biomarkers and confounding risk factors, thereby generating a validated stress-risk index;
vi) discriminating the AUC between the positives and negatives of Y using the predicted probability of positives and using the sensitivity and specificity of correct predictions as diagnostic for predictive validity;
vii) determining an optimal V cut-off value using ROC analysis;
viii) using a non-linear regression model that includes neural networks as comparison to the logistic regression model;
ix) determining the maximum of the Youden index using the ROC curves with the non-linear regression analysis substantiating a functional relationship between the models using multilayer perceptron with two layers and trained with Bayesian regularization, wherein hidden layers have tansig functions and an output layer is linear with ten bootstrap repetitions; and/or
x) optimizing the neural networks and extracting the functional relationship with analysis markers of a stroke risk profile of a patient.

The method can further include: xi) diagnosing a risk of the patient of developing chronic stress and ischemic heart disease related stroke, utilizing the validated stress-risk index for a positive prediction of the stroke risk profile of the patient; and/or xii) facilitating a therapeutic decision by authorized personnel (e.g., medical personnel, such as doctors, nurses, or the like) for the patient, based on the diagnosis of the risk of developing chronic stress and ischemic heart disease related stroke. The step of facilitating a therapeutic decision (step xii)) can comprise generating, by authorized personnel and based on the diagnosed risk of the patient of developing chronic stress and ischemic heart disease related stroke utilizing the validated stress-risk index, one or more preventative recommendations tailored to the patient. The one or more preventative recommendations can be provided to the patient for implementation to reduce the assessed stroke risk (e.g., the diagnosed risk of the patient of developing chronic stress and ischemic heart disease related stroke). The method can further comprise: xiii) treating the patient (e.g., with targeted/tailored preventative recommendations (such as the one or more preventative recommendations tailored to the patient), and the targeted/tailored preventative recommendations can be provided to the patient for implementation to reduce the assessed/diagnosed stroke risk). The targeted/tailored preventative recommendations (e.g., the one or more preventative recommendations tailored to the patient) can include pharmaceutical, diet, lifestyle, and/or exercise-based intervention, as deemed appropriate by the authorized personnel in view of the diagnosis. The biological samples obtained can be selected from the group consisting of, for example, blood, serum, plasma, urine, and saliva. The biological samples obtained can be, for example, fasting biological samples.

In another embodiment, a method for establishing a chronic stress and diabetes related stroke risk phenotype, wherein an adaptation of the UCLA stroke risk score was used to determine the risk of chronic stress and diabetes related stroke in an individual, can comprise:
i) using the stress-risk index determined as discussed above to establish a stress-d-risk index;
ii) performing a statistical analysis of biomarkers and risk factors, wherein variables with skewed non-normal distributions were logarithmically transformed, wherein from among the variables of the adapted UCLA score, the following nine were used as analysis markers: age, sex, systolic blood pressure, use of hypertensive drugs, smoking habit, diabetes, history of cardiovascular disease (e.g., coronary heart disease, cardiac failure, and/or intermittent claudication), electrocardiography (ECG) atrial fibrillation, and electrocardiography left ventricular hypertrophy (ECG-LVH), wherein adaptation of the UCLA stroke risk score included replacement of self-reported values of the analysis markers with quantitative markers including HbA1C≥6.5% as a marker for diabetes and nicotine metabolite cotinine≥14 ng/ml as a marker for smoking, wherein, in addition, the liver enzyme gamma glutamyl transferase (GGT) was added as a marker for alcohol abuse in developing the stress-d-risk index;
iii) determining standardized values of the analysis markers by principal component analysis (PCA) at baseline;
iv) computing the first principal component scores as a weighted mean of standardized variables with determined weights reflecting seven component loadings that are cotinine, GGT, diabetes defined as HbA1C≥6.5%, systolic blood pressure, perfusion deficits, ECG atrial fibrillation, and ECG-LVH;
v) determining the stress-d-risk index by multiplying the component scores values by ten and increasing it by fifty, such that a mean of the stress-d-risk index is 50 and its standard deviation lies between 0 and minus 100;
vi) determining a cut-point for the stress-d-risk index by conducting a ROC analysis using the cut-off determined in step iv) of claim 18 to discriminate between the positives and negative data and the sensitivity, specificity, and percentage of correct predictions;
vii) denoting a dichotomous variable, which discriminates between those respondents above the cut point and those below the cut point as Y, where the stress-d-risk index AUC was 0.78 (with a 95% CI of 0.73, 0.83) for a positive prediction (with 81% sensitivity and 59% specificity);
viii) validating the stress-d-risk index by applying multivariate linear regression analysis in a model using logarithmic transformed predictors in a complete dataset of N=349 by using subsets of 10 training sets with each 60% of population and 10 test sets with the remaining 40% of population;
ix) applying a logistic linear regression model by using logarithmic transformed predictors in all data as in step viii) using variable Y as in step vii);
x) validating the logistic linear regression model by repeating step ix) on 10 randomly selected samples and 10 test sets, thereby generating a validated logistic linear regression model;
xi) predicting a probability of risk for chronic stress and diabetes related stroke by obtaining a maximum likelihood estimates of regression coefficients of all regressions;
xii) using the dichotomous variable Y of the stress-d-risk index as in step vii) to discriminate between positives and negatives of the markers of step xi), wherein the stress-d-risk index AUC was 0.82 for a positive prediction;
xiii) using a logistic regression analysis wherein Y is used as the dependent variable and V contains the selected input continuous stress biomarkers as predictors of positives;
xiv) determining an optimal cut-off value for V using ROC analysis;
xv) using the AUC in the ROC analysis on V, using Y and the sensitivity, specificity, and percentage of correct predictions at the cut-off value as diagnostics for a predictive validity of V; and
xvi) performing Hosmer-Lemeshow tests for testing goodness of fit for the logistic linear regression model in all participants, training, and test sets; and
The method can further comprise: xvii) diagnosing a risk of the patient of developing chronic stress and diabetes related stroke, utilizing the validated logistic linear regression model; and/or xviii) facilitating a therapeutic decision for the patient by authorized personnel (e.g., medical personnel, such as doctors, nurses, or the like), based on the diagnosis of the risk of developing chronic stress and diabetes related stroke. The step of facilitating a therapeutic decision (step xviii)) can comprise generating, by authorized personnel and based on the diagnosed risk of the patient of developing chronic stress and diabetes related stroke utilizing the validated logistic linear regression model, one or more preventative recommendations tailored to the patient. The one or more preventative recommendations can be provided to the patient for implementation to reduce the assessed stroke risk (e.g., the diagnosed risk of the patient of developing chronic stress and diabetes related stroke). The method can further comprise: xix) treating the patient (e.g., with targeted/tailored preventative recommendations (such as the one or more preventative recommendations tailored to the patient), and the targeted/tailored preventative recommendations can be provided to the patient for implementation to reduce the assessed/diagnosed stroke risk). The targeted/tailored preventative recommendations (e.g., the one or more preventative recommendations tailored to the patient) can include pharmaceutical, diet, lifestyle, and/or exercise-based intervention, as deemed appropriate by the authorized personnel in view of the diagnosis. The biological samples obtained can be selected from the group consisting of, for example, blood, serum, plasma, urine, and saliva. The biological samples obtained can be, for example, fasting biological samples.

A person of ordinary skill in the art would understand, from the disclosure herein combined with knowledge within the art, how to calculate the adapted UCLA score (which is a 10-year stroke risk composite score of UCLA). The nine markers in the UCLA are the same as those in the Framingham Risk Study (FRS) in D'Agostino et al. (Stroke risk profile: Adjustment for antihypertensive medication, The Framingham study, Stroke, 1994, 25:40-43, doi: 10.1161/01.str.25.1.40; which is hereby incorporated herein by reference in its entirety). Among the variables of the adapted UCLA score, the following nine can be used as analysis markers-age, sex, systolic blood pressure, use of hypertensive drugs, smoking habit, diabetes, history of cardiovascular disease (e.g., coronary heart disease, cardiac failure, intermittent claudication), ECG-atrial fibrillation, and ECG-LVH. The FRS risk score in D'Agostino et al. included the following nine analysis markers-age, sex, systolic blood pressure, use of hypertensive drugs, smoking habit, diabetes, history of cardiovascular disease (coronary heart disease, cardiac failure, intermittent claudication), ECG-atrial fibrillation, and ECG-LVH (see also Wolf et al, Probability of stroke: a risk profile from the Framingham Study, Stroke, 1991 Mar. 22 (3): 312-8, doi: 10.1161/01.str.22.3.312, PMID: 2003301; which is hereby incorporated herein by reference in its entirety). Though, throughout Wolf et al. and D'Agostino et al., there is not necessarily consistency in the medical conditions mentioned for the analysis marker, namely history of cardiovascular disease (CVD).

Perfusion deficits as indicator of coronary artery disease (CAD), also known as coronary heart disease (CHD) or ischemic heart disease, refer to inadequate blood flow to the heart muscle itself. CHD encompasses conditions such as stable angina, acute coronary syndrome (ACS), and silent myocardial ischemia (see also, Shahjehan et al., Coronary Artery Disease, In: StatPearls [Internet], Treasure Island (FL), StatPearls Publishing, 2025, ncbi.nlm.nih.gov/books/NBK564304/; which is hereby incorporated herein by reference in its entirety). Cardiac failure and intermittent claudication (PAD) are both considered markers or clinical manifestations of perfusion defects in the peripheral arterial system (see also, Zemaitis et al., Peripheral Arterial Disease, In: StatPearls [Internet], Treasure Island (FL), StatPearls Publishing, 2025, ncbi.nlm.nih.gov/books/NBK430745/; which is hereby incorporated herein by reference in its entirety).

The adapted UCLA score can be calculated, for example with reference to D'Agostino et al. and/or Wolf et al. using D'Agostino et al. The UCLA 10-year stroke risk score calculator referenced the FRS (see also D'Agostino et al. and Wolf et al.). The adapted UCLA score can be calculated as follows by using the following predictors (see also, e.g., framinghamheartstudy.org/fhs-risk-functions/stroke/; which is hereby incorporated herein by reference in its entirety)—age, sex, systolic blood pressure, use of hypertensive drugs, diabetes, cigarette smoking, history of cardiovascular disease, ECG atrial fibrillation, and left ventricular hypertrophy (e.g., ECG-LVH).

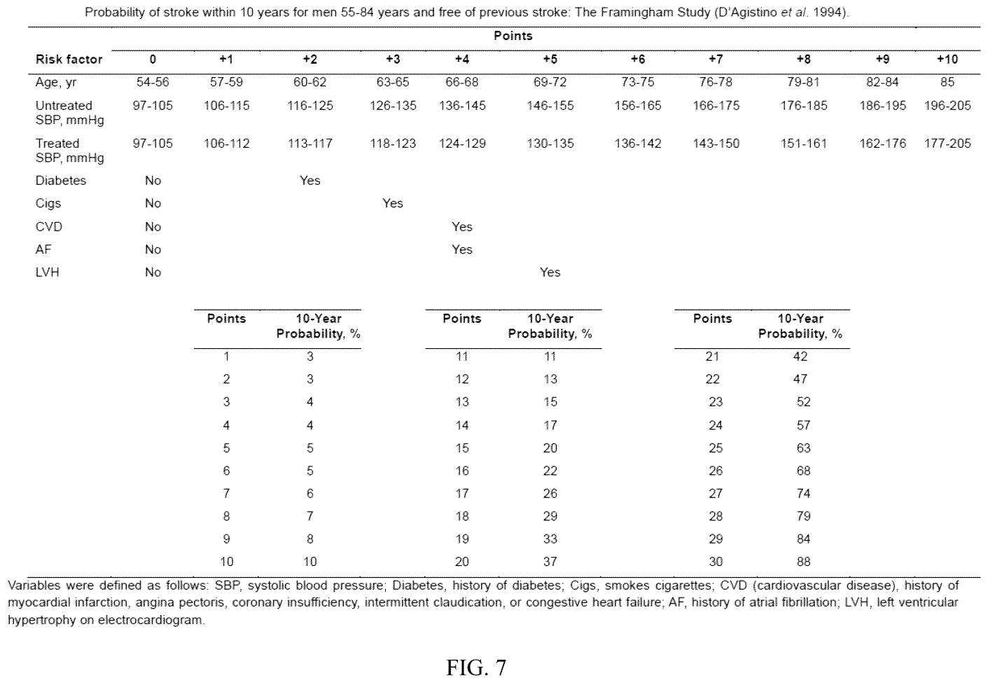

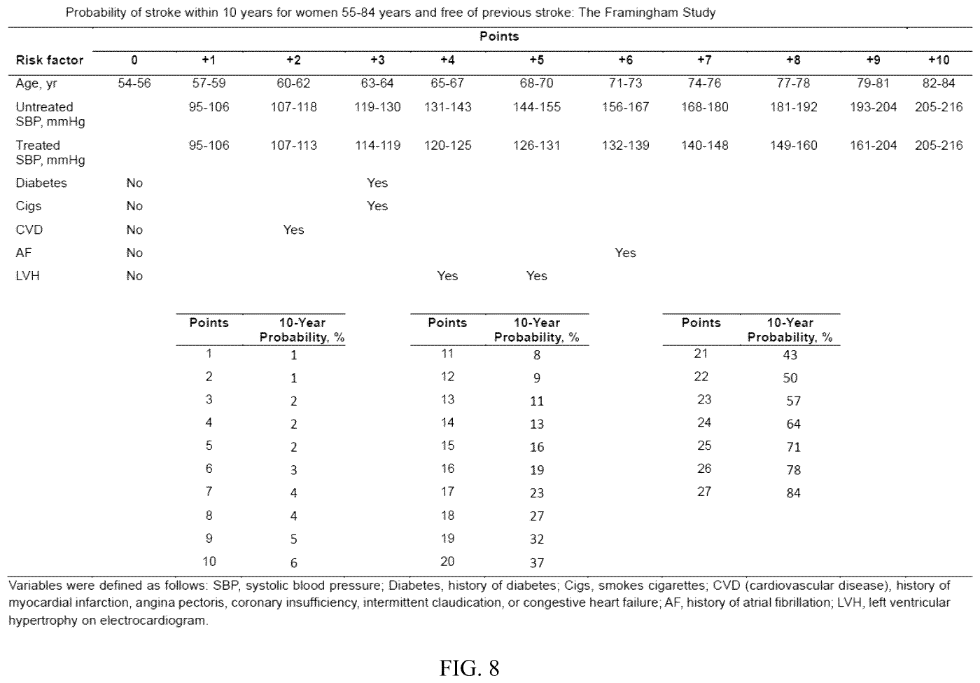

D'Agostino et al. and Wolf et al. developed the FRS to determine the probability of stroke by using a point system in sex-specific groups (see also the tables in FIGS. 7 and 8). Variables were defined as follows-systolic blood pressure (SBP) [=0 if SBP<110 or =10 if SBP>200]; use of hypertension medication (HTX) [=1 if on medication, =0 if not]; history of cardiovascular disease (coronary heart disease, cardiac failure, intermittent claudication) [yes=1, no-0]; cigarette smoking [yes=1, no=0]; diabetes [yes-1, no=0]; ECG-atrial fibrillation [yes=1, no-0]; left ventricular hypertrophy on electrocardiogram (ECG-LVH) [yes-1, no=0].

For example, a score of 16 points in a man yields a 10-year stroke probability of 22% (see FIG. 7); whereas a score of 16 points in a woman yields a 10-year stroke probability of 19% (see FIG. 8).

### Example 1

### Identification of Biomarkers

### Study Populations

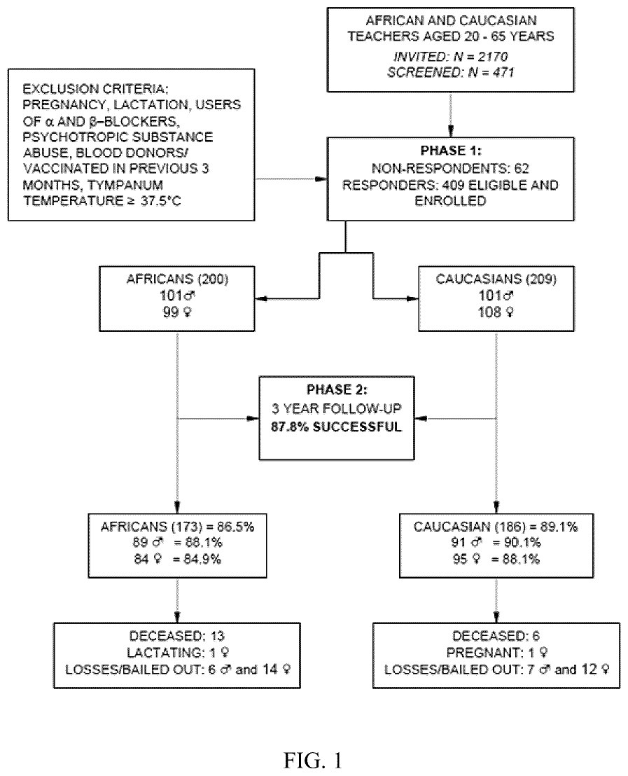

The target population (N=2170) including urban-dwelling well-educated Black (African) and White African (Caucasian) male and female teachers, enrolled in the 43 schools of the Dr Kenneth Kaunda Education District (Klerksdorp and Potchefstroom), North-West Province, South Africa, and were invited to participate (FIG. 1) in the Sympathetic activity and Ambulatory Blood pressure in Africans (SABPA) study cohort. All volunteering teachers had medical aid benefits and were screened to meet study eligibility criteria during the recruitment phase (FIG. 1). Those complying formed the respondent group of 409 (FIG. 1), and those not complying formed the non-respondent group (N=62) (FIG. 1). The Black teachers preferred to be informed and recruited in separate sex groups and the protocol, especially the amount of blood drawn and hair sampling, was not well received. Time constraints were the main obstacle for participation in the Caucasian teachers' cohort and mixed-sex informed recruitment sessions were not a problem. Data is currently available for 409 teachers of Phase I from which 359 were followed up in Phase II. Exclusion criteria were pregnancy, lactation, tympanum temperature≥37.5° C., the use of psychotropic substances or α- and β-blockers, and blood donors or individuals vaccinated within 3 months prior to data collection.

SABPA analyses were done at the North-West University, Potchefstroom. Facilities and equipment were available to receive and store fasting biological samples at −80° C., before performing analytical assays to detect risk markers.

### Established Risk Factors

An adaptation of the University of California, Los Angeles 10-year stroke risk composite score (UCLA) [American Heart and Stroke certified UCLA Medical Centre, Primary Stroke Centre, Santa Monica, Los Angeles, USA] was deemed necessary to establish a chronic stress and stroke risk phenotype and said UCLA includes the following variables: individual's medical history (i.e. cardiovascular disease, kidney disease, myocardial infarction, diabetes and hypertension medication usage), demographic and lifestyle factors (age, race, sex, diabetes, smoking, alcohol use and physical activity habits), systolic and diastolic blood pressure, fibrinogen, waist circumference, perfusion deficits (myocardial ischemia), electrocardiography (ECG) atrial fibrillation and ECG left ventricular hypertrophy.

### Biomarkers

Fasting blood samples were collected before 09:00 in the morning to avoid circadian rhythm response fluctuations. Samples were handled according to standardized procedures and frozen at −80° C. until required for analysis. For the proposed biochemical analyses, a serum/plasma/urine sample of 500 μl and plasma sample of 200 μl were needed. If serum or plasma was used, the dead volume when using the Hitachi cups for electrochemiluminescence immunoassays on the e411 (ROCHE, Basel, Switzerland) was considered for biochemical analyses.

A registered nurse collected fasting blood samples. All biochemical analyses were done in duplicate on never thawed serum, plasma, urine or saliva samples. Serum cotinine values (indicative of smoking) were derived from a homogeneous immunoassay (Modular ROCHE Automized systems, Basel, Switzerland). Serum and whole blood EDTA samples were analyzed for gamma glutamyl transferase (GGT as indicator of alcohol use), lipids and high sensitivity c-reactive protein (CRP) with an enzyme rated method (Enzymatic colorimetric assay, Cobas Integra 400 plus, ROCHE, Basel, Switzerland. Whole blood EDTA glycated hemoglobin (HbA_{1C}) was analysed with turbidimetric inhibition immunoassays (Cobas Integra 400 Plus, ROCHE Basel, Switzerland). Citrate fibrinogen values were derived by using the viscosity-based clotting method Immuno-turbimetric method 540 nm (Instrument: STA Compact; TAGO Diagnostic, ROCHE, France). Saliva cortisol was analysed with an electrochemiluminescence immunoassay kit (Catalogue number DE2989; Demdemitic Diagnostics GmbH, Kiel-Welsee, Germany). Serum cortisol, ACTH, and high sensitivity cardiac troponin were determined with an electrochemiluminescence immunoassay (ECLIA), Elecsys 2010 (ROCHE Basel, Switzerland). Values below detectable limit were substituted with lower than detectable values using log-methods. Urine collection was performed overnight, 8 h sampling at baseline and 24 h sampling at follow-up (Malan et al., 2017). At follow-up, participants began and ended sampling with an empty bladder on Day 1. Urine was collected for the next 24 h in a three liter container, washed with 9 ml of 20% HCl (UriSet24, Sarstedt®, Nümbrecht, Germany). Samples were stored at −80° C. until analysis within one year after collection, using the 3-Cat Urine ELISA Fast Track kits (SKU: BA E-6600, LDN, Nordhorn, Germany) where a standard range of 0.5-1000 ng/ml was reported. Intra- and inter-assay coefficients for epinephrine were 5.50% and 9.62% respectively and for norepinephrine, 2.70% and 8.59%. Urine creatinine was measured using the enzymatic method (COBAS Integra 400 Plus, ROCHE, Basel; Switzerland). Intra- and inter-assay coefficients for all biochemical analyses were below 10%.

### Cardiovascular Measurements

A combined ambulatory blood pressure-electrocardiogram apparatus (Cardiotens CE120®, Meditech, Budapest Hungary) was applied between 07:00-09:00 on working days (Monday-Thursday) at the teachers' school of employment. The blood pressure cuff was fitted to the non-dominant arms using an appropriate cuff size. Blood pressure measures were obtained every 30 minutes during the day (08:00-22:00) and hourly during the night (22:00-06:00). Participants continued their usual daily activities and were asked to record occurrences of stress, physical activity, headache, syncope, dizziness, nausea, palpitations, hot flushes and visual disturbances on their ambulatory diary card. The 24 h successful inflation rate was 77.9% (±12.9) in “Stressed” individuals and 81.8% (±10.1) in “no-Stressed” individuals. The data were analyzed with the CardioVisions 1.19 Personal Edition software (Meditech, Budapest, Hungary). Hypertensive status was classified as 24 h SBP≥130 mm Hg and/or DBP≥80 mm Hg (European Society of Cardiology, 2018). Participants resumed their normal school and extra-curricular activities till 15:00 and hereafter transported to the North-West University for clinical measures. They fasted from 22:00 till 07:00 when anthropometric measures according to standardized as well as blood samples were collected followed by physical activity measures (Malan et al., 2015).

Silent myocardial ischemia (SMI) events or perfusion deficits: were assessed by two-channel 24 h electrocardiogram (ECG) recordings (Cardiotens CE120®, Meditech, Budapest, Hungary) for 20 seconds at 5 minute intervals. Before the start of the ambulatory investigation, the isoelectric reference point (PQ segment), J point, L point (80 ms after the J point), and an ST-segment detection interval of at least 3 mm as the initial ST level, were calculated individually for each patient. An ischemic event was recorded according to the following criteria: horizontal or descending ST-segment depression by at least 1 mm; duration of the ST-segment episode lasting ≥1 minute, and a ≥1-minute interval from the preceding episode. In case of a horizontal or descending ST depression (1 mm-1 minute duration at a 1 minute interval from the preceding episode), an ECG tracing lasting 60 seconds was recorded and an additional blood pressure measurement was automatically initiated by the trigger mechanism of the device.

A resting 12-lead ECG (strip lead II) was used to identify atrial fibrillation cases and which were confirmed by a medical practitioner (NORAV Medical Ltd PC 1200, software version 5.030, Israel). A 12-lead ECG determined ECG left ventricular hypertrophy using strip leads RaVL+SV3 in the calculation of a gender-specific formula, the Cornell product: sum of leads (RaVL+SV3)*QRS>244 mV·ms.

### Retinal Vessel Analyses (Stroke Risk Marker) (Malan et al., 2020)

Mydriasis was induced in the right eye of the participant by means of a drop containing tropicamide, 1% and benzalkonium chloride 0.01% (m/v). Fundus imaging was performed in a well-controlled light and temperature regulated room with the retinal vessel analyser with a Zeiss FF450^{Plus} camera and the software VesselMap 2, Version 3.02 (Imedos Systems GmbH, Jena, Germany). Retinal vessel calibres were measured as monochrome images by manually selecting first order vessel branches in a measuring zone located between 0.5 and 1.0 optic disc diameters from the margin or the optic disc. Upon selection of the vessel, software automatically delineated the vessels' measuring area. A color image was used as reference to ascertain correct identification of arteries and veins and two experienced scientists agreed on the vessel type before selection. Reproducibility was computed for a randomly selected cohort with a correlation coefficient of 0.84. Diastolic ocular perfusion pressure (DOPP) measures were obtained as hypo-perfusion risk marker in the microvasculature. A local anesthetic drop (Novasine Wander 0.4% Novartis) was inserted in the right eyes in 99% of all cases to measure intra-ocular pressure (IOP) with the Tono-Pen Avia Applanation Tonometer (Reichert 7-0908, ISO 9001, New York, USA). DOPP was calculated (DBP minus intra-ocular pressure) and hypertensive/diabetic retinopathy was diagnosed by a registered ophthalmologist.

### Statistical Analysis

A risk score for chronic stress and ischemic heart disease related stroke (herein referred to as STRESS^{risk} index) may reflect chronic stress and stroke risk. The statistical software packages used were Statistica version 13.3 (TIBCO Software Inc., Palo Alto, USA, 2018); IBM SPSS version 23 statistical and SAS® 9.4 (Statistical Analysis System). Variables with skewed distributions were log-transformed. The statistical significance level was set at p≤0.05 (two-tailed).

The STRESS^{risk} index was determined as follows:

A multiple stepwise linear regression of biomarkers and risk factors (transformed to be normally distributed) of the UCLA was performed.

The Receiver Operating Characteristic (ROC) analysis is commonly used to assess the difference between two distributions (binary classification) at all classification thresholds. The ROC space consists of a plot of a continuous system represented by a (ROC) curve, created by plotting the true positive rate against the false positive rate, and the area under the ROC curve (AUC) is employed as a measure of the performance of the predictions made from the classification system across the different thresholds. The STRESS^{risk} index, for three or more possible combination of continuous biomarkers, was determined as the AUC as a maximum (Youden index: sensitivity+specificity−1) when discriminating the positive and negatives of the UCLA composite dichotomous marker (range from 2-30%) denoted as Y. The Youden index is a method that finds the point on the ROC curve farthest from the change classification, and is used to identify a “optimal” cut-off value. Here, the term “optimal” refers to the cut point that maximizes correct classifications and/or minimizes incorrect classifications. Accordingly, an optimal cut-off value for the STRESS^{risk} index was used and denoted as biomarker V.

### Validation of the Biomarker V:

Here Y was used as the dependent variable and V as predicted probability of positives using a logistic regression on the selected input continuous biomarkers and confounding risk factors.

To discriminate the AUC between the positives and negatives of Y using the predicted probability of positives and also the sensitivity and specificity of correct predictions were used as diagnostics for predictive validity. An optimal cut-off value was further determined for V using the ROC analysis.

Non-linear regression model, that includes neural networks, was compared with the logistic regression model. The maximum of the Youden index (sensitivity+specificity−1) was determined using the ROC curves, with the non-linear regression analyses substantiating the novel functional relationship between the models using multilayer perceptron with 2 layers and trained with Bayesian regularization. Hidden layers have tansig functions and the output layer is linear with 10 bootstrap repetitions.

Once the networks were optimized, they were used to extract the required functional relationships with the UCLA stroke risk scores.

### Results

TABLE 1
Clinical characteristics of a chronic stress and ischemic heart disease related stroke risk phenotype.

| | Stressed | | Non-Stressed | | |
|---|---|---|---|---|---|
| | (N = 236) | | (N = 123) | | P-values |
| Age, yrs | 44.5 | (39.0-51.0) | 47.0 | (41.0-54.0) | 0.04 |
| Women, n (%) | 88 | (52.1) | 82 | (47.4) | 0.39 |
| Urban living, years | 31.8 | (19.0-45.0) | 20.5 | (10.0-30.0) | <0.001 |
| Cotinine, ng/ml | 0.01 | (0.01-15.51) | 0.01 | (0.01-0.01) | 0.33 |
| GGT, U/l | 43.5 | (28.4-74.4) | 18.0 | (12.0-28.0) | <0.001 |
| Physical activity, kcal/24 h | 2584.6 | (2185.9-3118.1) | 2968.0 | (2370.0-3540.7) | <0.001 |
| Waist circumference, cm | 98.2 | (88.7, 106.3) | 83.3 | (74.6, 93.4) | <0.001 |

Ischemic heart disease and stroke risk markers

| | | | | | |
|---|---|---|---|---|---|
| Thyroid stimulating hormone, μIU/ml | 1.8 | (1.3-2.5) | 2.1 | (1.4-2.9) | 0.01 |
| Intra-ocular pressure (mmHg) | 16 | (4) | 15 | (4) | 0.044 |
| Retinal artery caliber (MU) | 148.5 | (11.1) | 154.1 | (13.4) | <0.001 |
| Retinal vein caliber (MU) | 243.2 | (20.0) | 240.6 | (20.2) | 0.267 |
| Retinopathy, n (%) | 134 | (57) | 55 | (45) | 0.024 |
| Cardiac Troponin T, ng/L | 4.2 | (3.1-5.5) | 4.9 | (3.2-6.9) | 0.05 |
| Cholesterol, mmol/l | 4.5 | (3.8-5.5) | 5.5 | (4.7-6.4) | <0.001 |
| CRP:Fibrinogen, g/L:mg/L | 1.4 | (0.7-2.6) | 0.5 | (0.4-1.2) | <0.001 |
| 24 h SBP, mm Hg | 131 | (122-143) | 124 | (116-130) | <0.001 |
| 24 h DBP, mm Hg | 82 | (77-90) | 77 | (71-82) | <0.001 |
| 24 h Heart rate, bpm | 79 | (73-86) | 74 | (68-81) | <0.001 |
| 24 h Hypertension, n (%) | 176 | (75) | 19 | (16) | <0.001 |
| 24 h urinary NE:Cr | 18.8 | (11.6-29.8) | 24.8 | (13.2-38.9) | 0.07 |
| 24 h urinary E:Cr | 2.9 | (1.6-2.9) | 2.9 | (1.6-4.7) | 0.36 |

Medications, n (%)

| | | | | | |
|---|---|---|---|---|---|
| Statins | 2 | (1.2) | 6 | (3.5) | 0.16 |
| Aspirin | 4 | (2.4) | 9 | (5.2) | 0.17 |
| ACE inhibitors | 19 | (11.2) | 3 | (1.7) | <0.001 |
| Angiotensin II blockers | 1 | (0.6) | 1 | (0.6) | 0.99 |
| Diuretics | 23 | (13.6) | 8 | (4.6) | <0.001 |
| Calcium channel blockers | 13 | (7.7) | 1 | (0.6) | 0.001 |
| Beta blockers | 5 | (3.0) | 1 | (0.6) | 0.09 |

| | | | |
|---|---|---|---|
| Alpha blockers | 0.0 | 0.0 | — |

Independent t-tests were used to compare “Stressed” vs. “non-Stressed” groups.
Values are mean (±SD), median (±interquartile range/IQR) or frequencies (%).
Where: GGT, gamma glutamyl transferase; CRP, C-reactive protein; HDL, high density lipoproteins; HRV, heart rate variability measuring the standard deviation of the 12-lead ECG RR interval; NE:Cr, norepinephrine creatinine ratio; E:Cr, epinephrine creatinine ratio. Hypertensive status classified as 24 h SBP ≥130 mm Hg and/or DBP ≥80 mm Hg.

### Example 2

The retinal vessels offer an easily accessible view of the vasculature, which might reflect emotional stress pathology and stroke risk. As the retina shares embryonic origins with the brain, with similar anatomy and blood-barrier physiology; it is of particular interest as a marker of cerebrovascular and neurodegenerative disease.

The STRESS^{risk} index was used to assess “Stressed” vs. “non-Stressed” groups, independent of race or sex. Findings showed that behavior and biological processes are tightly interlinked in the brain-retina-heart axis. For example, cardiac injury and stroke risk markers (including retinal arterial narrowing) reflected inflammation and oxygen perfusion deficits, of which is associated with increased risk for stroke in “Stressed” individuals. Furthermore, arterial narrowing and vein widening in the retina were associated with decreased glial cell functioning in these “Stressed” individuals (Malan et al., 2020), which resembles findings previously shown in the prefrontal cortex of suicide cases and severely depressed patients. As a consequence, this may also have debilitating effects on retinal ganglion cell health and visual function.

The emerging metabolic perturbation and endothelial dysfunction observed in the “Stressed” individuals is determined as the chronic stress and diabetes related stroke risk phenotype (herein referred to as STRESS^{d-risk}).

### Study Population

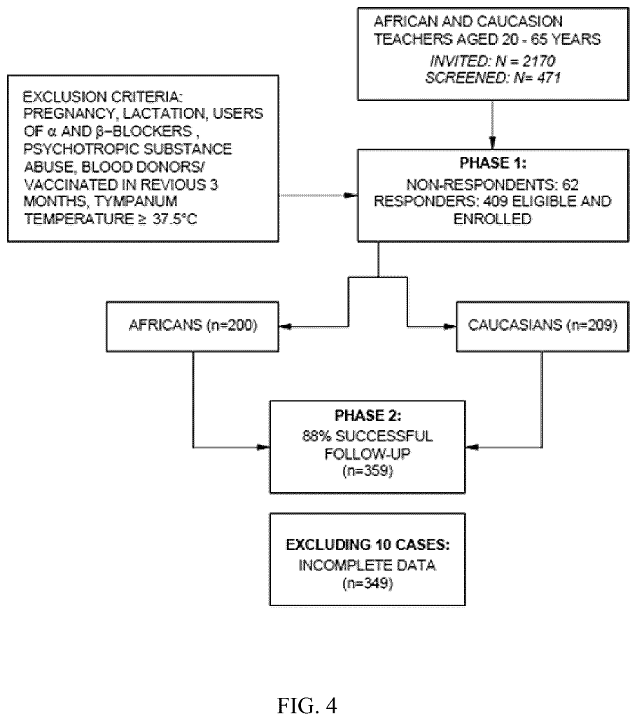

The SABPA prospective cohort study was used as complete data source (n=349) (FIG. 4) and all individuals participated at baseline and at 3-year follow-up. The rationale for the selection of the participants was to obtain a sample from a homogenous working environment with similar socio-economic status. Seasonal changes were avoided and extensive clinical assessments were to be performed in a well-controlled temperature and light setting environment. Exclusion criteria at baseline were pregnancy, lactation, tympanum temperature≥37.5° C., the use of psychotropic substances or α- and β-blockers and blood donors or individuals vaccinated within 3 months prior to data collection. Participants were fully informed about the objectives and procedures prior to recruitment and provided written, informed consent.

### Biochemical Analyses

Participants were in a semi-recumbent position for at least 30 minutes before 09:00 in both study phases. A registered nurse obtained fasting blood samples from the antebrachial vein branches of the dominant arm of each participant with a winged infusion set. All blood samples were handled according to standardized procedures and stored at −80° C. until analyses. All biochemical analyses were done in duplicate on never thawed serum/plasma samples. Serum cotinine values (indicative of smoking) were derived from a homogeneous immunoassay (Modular ROCHE Automized systems, Basel, Switzerland). Serum and whole blood EDTA samples were analyzed for gamma glutamyl transferase (GGT as indicator of alcohol use), lipids and high sensitivity c-reactive protein (CRP) with an enzyme rated method (Enzymatic colorimetric assay, Cobas Integra 400 plus, ROCHE, Basel, Switzerland. Total Insulin-like growth factor-1 was determined in serum using an immunoradiometric assay (IRMA) from Immunotech, Beckman Coulter (A15729). With an inter-assay percentage coefficient of variation of 4.49 and an intra-assay percentage coefficient of variation 2.92. Whole blood EDTA glycated hemoglobin (HbA_{1C}) were analysed with turbidimetric inhibition immunoassays (Cobas Integra 400 Plus, ROCHE Basel, Switzerland). The American Diabetes Foundation guidelines were used to define pre-diabetes status as ≥5.7%; diabetes as HbA_{1C}≥6.5%; and HOMA-IR by using the following formula: fasting glucose×fasting insulin/405 [normal IR, <3, moderate IR, 3-5; and severe IR, >5].

### Statistical Analysis

The statistical software packages used were Statistica version 13.3 (TIBCO Software Inc., Palo Alto, USA, 2018); IBM SPSS version 23 statistical and SAS® 9.4. The SABPA prospective cohort study was used as complete data source (n=349) and all individuals participated at baseline and at 3-year follow-up.

The following statistical analyses were carried out in order to validate chronic stress and determine the probability of diabetes related stroke risk.

Variables with skewed non-normal distributions were logarithmic transformed. The statistical significance level was set at p≤0.05 (two-tailed).

The validated chronic stress risk biomarkers predictive of stroke were used to establish a chronic stress and diabetes related stroke risk phenotype.

An adaptation of the UCLA was used to determine the risk of chronic stress and diabetes related stroke risk in an individual, where the 9 independent markers of said UCLA include: age, sex, systolic blood pressure, self-reported use of hypertensive drugs, diabetes, smoking habit, perfusion deficits (myocardial ischemia), ECG atrial fibrillation and ECG-LVH. The UCLA includes self-reported measures for use of hypertensive drugs, diabetes and smoking habit, which may not be reliable due to concerns relating to biases and other limitations (Epel et al., 2018; Malan et al., 2017; Malan et al., 2020). Accordingly, these self-reported variables were replaced with quantitative validated markers, i.e. HbA_{1C}≥6.5% as a marker for diabetes (American Diabetes Association 2019) and nicotine metabolite (i.e. cotinine≥14 ng/ml) as a marker for smoking. In addition, alcohol abuse, did not form part of the UCLA and thus the liver enzyme, GGT, was used as a marker for alcohol abuse (Hastedt et al., 2016; Enhörning & Malan 2019). Apart from high blood pressure, habitual consumption of large amounts of alcohol is one of the most important risk factors for stroke (Solveig et al., 2018), contributing to more than 50% of all stroke cases in the United Kingdom. Furthermore, a previous study has shown that alcohol abuse was associated with high blood pressure (Hamer et al., 2011), which is also related with the increased risk of developing ischemic heart disease (Oosthuizen et al., 2016; Wentzel et al., 2018) as well as the onset of stroke (Mostofsky et al., 2010).

Standardized values of the adapted UCLA were determined by using principal component analysis at baseline. The first principal component scores were computed as a weighted mean of standardized variables with determined weights reflecting 7 component loadings [cotinine, GGT, diabetes (HbA1C≥6.5%), systolic blood pressure, perfusion deficits (myocardial ischemic events), atrial fibrillation and ECG-LVH]. The first principal component scores then had a mean of 0 and standard deviation of 1. To obtain a convenient index, the component score values were multiplied by 10 and increased by 50. A so-called T-index, having a mean of 50 and a standard deviation of 10 and lies between 0 and −100, was denoted as the STRESS^{d-risk} index.

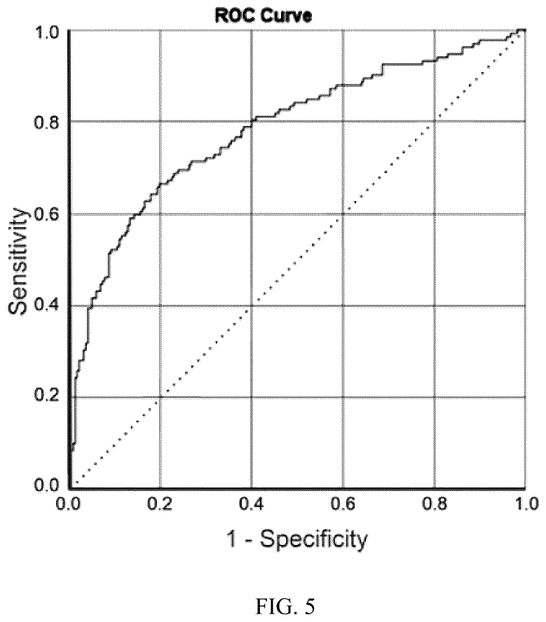

A cut-point for STRESS^{d-risk} index (discriminatory analyses) was determined by conducting the ROC analysis using the cut-off of the original UCLA 10-year stroke risk score (FIG. 5). This discriminated between the positive and negative data using the STRESS^{d-risk} index and also the sensitivity, specificity and percentage of correct predictions for obtaining the cut point. The dichotomous variable, which discriminates between those respondents who are at risk (when above the cut point) and those not at risk (below the cut point), is denoted by Y. The AUC was computed with 95% confidence limits.

Multivariate linear regression analysis was applied in a model using logarithmic transformed predictors to validate the chronic stress and diabetes related stroke risk phenotype (i.e. STRESS^{d-risk} index) in a complete dataset of N=349, by using subsets of 10 training sets (N=209, i.e. each 60% of population) and 10 test sets (the remaining 40%). Regression coefficient estimates and p-values were determined in all regressions.

Logistic linear regression was applied in a model by using logarithmic transformed predictors in all data as before using the aforementioned dependent variable Y. To validate the logistic linear regression model, model fitting was repeated on 10 randomly selected samples (training sets, each 60% of population) and 10 test sets (the remaining 40%). The maximum likelihood estimates of regression coefficients were obtained in all these regressions to predict the probability of risk for chronic stress and diabetes related stroke.

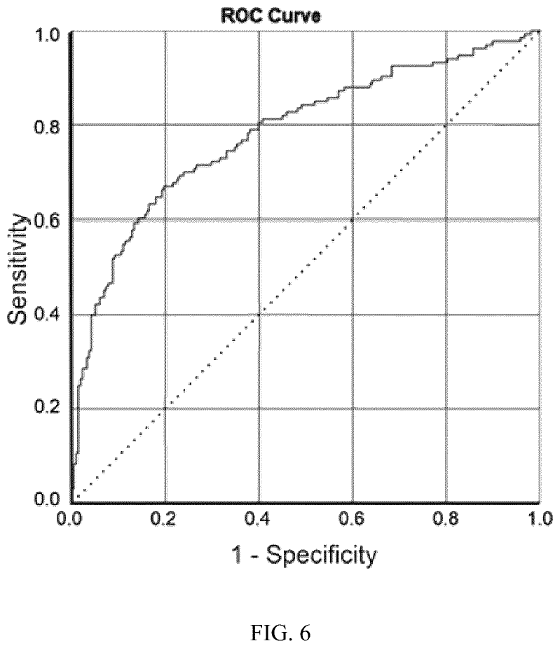

To discriminate between the positive and negatives of the novel stroke risk marker, the dichotomous marker Y of the STRESS^{d-risk} index was used. Using a logistic regression analysis, Y was used as the dependent variable and V, contained the selected input continuous stress biomarkers, as predictors of positives. An optimal cut-off value was further determined for V using ROC analysis (FIG. 6).

The AUC in the ROC analysis on V (using Y) and the sensitivity, specificity and percentage correct predictions at the cut-off value were considered as diagnostics for predictive validity of V. Hosmer-Lemeshow tests were performed to test the goodness of fit for the logistic regression risk prediction models (in all participants, 10 training and 10 test sets).

### Results

TABLE 2
Clinical characteristics of a chronic stress and diabetes related stroke risk phenotype.

| | Stress | | no-Stress | | |
|---|---|---|---|---|---|
| | (n = 159) | | (n = 105) | | P values |
| Age (years) | 46.2 | (±9.1) | 45.4 | (±9.2) | 0.501 |
| Ethnicity: Blacks | 97 | (61) | 21 | (20) | <0.001 |
| Sex: Men | 114 | (72) | 29 | (28) | <0.001 |

Diabetes and stroke risk markers

| | | | | | |
|---|---|---|---|---|---|
| Total energy expenditure (kcal/24 h) | 3145 | (±1706) | 2744 | (±807) | 0.015 |
| Smoking status | 45 | (28) | 6 | (6) | <0.001 |
| Alcohol abuse | 90 | (57) | 4 | (4) | <0.001 |
| Ethnic-specific central obesity | 105 | (66) | 54 | (34) | 0.018 |
| Dyslipidemia | 77 | (48) | 34 | (32) | 0.011 |
| Low grade inflammation | 86 | (54) | 40 | (38) | 0.011 |
| Insulin-like growth factor-I (ng/ml) | 0.9 | (0.1, 30.2) | 2.5 | (0.4, 64.9) | 0.031 |
| Pre-diabetes | 108 | (68) | 18 | (17) | <0.001 |
| Diabetes | 20 | (13) | 0 | (0) | <0.001 |
| Severe HOMA-IR | 52 | (33) | 11 | (11) | <0.001 |
| Perfusion deficits-DOPP (mmHg) | 73 | (±11) | 66 | (±11) | <0.001 |
| Diabetes/Hypertensive retinopathy | 98 | (62) | 46 | (44) | <0.001 |
| 24 h Hypertension | 118 | (74) | 32 | (31) | <0.001 |

Self-reported medication

| | | | | | |
|---|---|---|---|---|---|
| Diabetes | 8 | (5) | 0 | (0) | 0.020 |
| Hypertension | 48 | (30) | 18 | (17) | 0.021 |
| Anti-depressant | 1 | (1) | 1 | (1) | 0.767 |

Values are presented as mean (±SD), or N (%), and/or median (±95% interquartile range).
Abbreviations: Smoking status, cotinine, ≥14 ng/ml; Alcohol abuse, GGT ≥55 U/L (Oosthuizen et al., 2016); Ethnic-specific central obesity cut points (Malan et al., 2015); Dyslipidemia, total cholesterol:high density lipoprotein cholesterol ≥5.1; Low grade inflammation (CRP ≥3 ng/ml); Prediabetes, HbA1C ≥5.7%; Diabetes, HbA1C ≥6.5%; Severe HOMA-IR, homeostasis model of insulin resistance/IR assessment (>5); DOPP, Diastolic ocular perfusion pressure; 24 h Hypertension, SBP ≥130 and/or DBP ≥80 mmHg;

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein (including those in the References section) are incorporated by reference in their entirety, including all figures and tables, to the extent they are not inconsistent with the explicit teachings of this specification.

### REFERENCES

D'Agostino, R. B., Wolf, P. A., Belanger, A. J., Kannel, W. B. (1994). Stroke risk profile: Adjustment for antihypertensive medication, The Framingham study. Stroke 25, 40-43.
Enhörning, S., Malan, L. (2019). Copeptin relates to a fatty liver and measures of obesity in a South African population with mixed ethnicities. Endocrine 65, 304-311.
Epel, S. E., McEwen, B., Seeman, T., Matthews, K., Castellazo, G., Brownell, K. D., Bell, J., Ickovics, J. R. (2000). Stress and Body Shape: Stress-Induced Cortisol Secretion Is Consistently Greater Among Women With Central Fat. Psychosom Med 62, 623-632.
Hamer, M., Malan, L., Malan, N. T., Schutte, A. E., Huisman, H. W., van Rooyen, J. M., Schutte, R., Fourie, C. M. T., Seedat, Y. K. (2011). Conventional and behavioural risk factors and sub-clinical atherosclerosis in black and white Africans: The SABPA study. Atherosclerosis 215, 237-242.
Hastedt M, Büchner M, Rothe R, Gapert R, Herre S, Krumbiegel F, Tsokos M, Kienast T, Heinz A, Hartwig S. (2013). Detecting alcohol abuse: traditional blood alcohol markers compared to ethyl glucuronide (EtG) and fatty acid ethyl esters (FAEEs) measurement in hair. Forensic Sci Med Pathol 9 (4), 471-477.
Malan, L., Hamer, M., Frasure-Smith, N., Steyn, H. S., Malan, N. T. (2015). COHORT PROFILE: Sympathetic activity and Ambulatory Blood Pressure in Africans (SABPA) Prospective Cohort Study. Int J Epidem 44 (6), 1814-1822.
Malan, L., Hamer, M., von Känel, R., Lambert, G. W., Delport, R., Steyn, H. S., Malan, N. T. (2017). Chronic defensiveness and neuroendocrine dysregulation reflect a novel cardiac Troponin T cut point: the SABPA study: Psychoneuroendocrinology 85, 20-27.
Malan, L., Hamer, M., von Känel, R., van Wyk, R. D., Wentzel, A., Steyn, H. S., van Vuuren P, Malan N T. (2020). Retinal-glia ischemia and inflammation induced by chronic stress: the SABPA study. Brain, Behav Imm—Health 2, 100027.
Mirza, S. S., de Bruijn, R. F. A. G., Koudstaal, P. J., van den Meiracker, A. H., Franco, O. H., Hofman, A., Tiemeier, H., Ikram, A. (2016). The N-terminal pro B-type natriuretic peptide, and risk of dementia and cognitive decline: a 10-year follow-up study in the general population. J Neurol Neurosurg Psych, 87, 356-362.
Mostofsky, E., Burger, M. R., Schlaug, G., Mukamal, K. J., Rosamond, W. D., Mittleman, M. A. (2010). Alcohol and acute ischemic stroke onset: the stroke onset study. Stroke 41 (9), 1845-1849.
Oosthuizen, W., Malan, L., Scheepers, J. D., Cockeran, M., Malan, N. T. (2016). The defense response and alcohol intake: A coronary artery disease risk? The SABPA Study. Clin Exp Hypertens 38 (6), 526-532.
Solveig, A., Cunningham, M., Judd, S. E., Matz, L. M., Kabagambe, E. K., Moy, C. S., Howard, V. J. (2018). Alcohol Consumption and Incident Stroke Among Older Adults. J Gerontol B Psychol Sci Soc Sci 73 (4), 636-648.
von Känel, R., Hamer, M., Van Der Westhuizen, F. H., Malan, N. T., Malan, L. (2014). Leukocyte telomere length and hemostatic factors in a South African cohort: The SABPA Study. Thromb Hemo 12, 1975-1985.
Wentzel A, Malan L, Scheepers J D, Malan N T. (2018). QTc prolongation, increased NT-proBNP and pre-clinical myocardial wall remodelling in excessive alcohol consumers: The SABPA study. Alcohol 68, 1-8.
Wesseling, K. H., Jansen, J. R. C., Settels, J. J., Schreuder, J. J. (1993). Computation of aortic flow from pressure in humans using a nonlinear, three-element model. J App Physiol 74, 2566-2573.
Wolf, P. A., D'Agostino, R. B., Belanger, A. J., Kannel, W. B. (1991). Probability of stroke: a risk profile from the Framingham Study. Stroke. 22, 312-318.

What is claimed is:

1. A method for determining a stress-risk index for the risk of developing chronic stress and ischemic heart disease related stroke, wherein said method comprises:
i) performing a multiple stepwise linear regression of biomarkers and risk factors, both transformed to be normally distributed, of an adapted UCLA score, wherein said adapted UCLA score is a 10-year stroke risk composite score of the University of California, Los Angeles (UCLA), and wherein the adapted UCLA score includes as variables: individual's medical history regarding any cardiovascular disease, kidney disease, myocardial infarction, diabetes, and hypertension medication usage; demographic and lifestyle factors including age, race, sex, diabetes, smoking, alcohol use, and physical activity habits; systolic and diastolic blood pressure; fibrinogen; waist circumference; perfusion deficits including myocardial ischemia; electrocardiography atrial fibrillation; and electrocardiography left ventricular hypertrophy; wherein the biomarkers and risk factors have been determined by obtaining biological samples from individuals; measuring levels of the biomarkers in the biological samples obtained from the individuals and measuring levels of clinical markers of the individuals, wherein the biomarkers are serum cotinine values, gamma glutamyl transferase (γ-GT), lipids and high sensitivity c-reactive protein (CRP), whole blood EDTA glycated hemoglobin (HbA1C), citrate fibrinogen values, saliva cortisol, serum cortisol, adrenocorticotrophic hormone (ACTH), high sensitivity cardiac troponin, urinary norepinephrine, epinephrine, and creatinine, wherein the clinical markers are systolic blood pressure (SBP), diastolic blood pressure (DBP), silent myocardial ischemia (SMI) events or perfusion deficits, electrocardiography (ECG) atrial fibrillation, ECG left ventricular hypertrophy (ECG-LVH), retinal vessel calibers, intra-ocular pressure (IOP), and diastolic ocular perfusion pressure (DOPP) calculated from DBP minus IOP;
ii) performing a receiver operating characteristic (ROC) analysis to assess a difference between the distribution of biomarkers and the distribution of risk factors at all classification thresholds;
iii) determining a stress-risk index for three or more possible combinations of the biomarkers as an area under the curve (AUC) as a maximum of the ROC analysis, when discriminating positives and negatives of a composite dichotomous biomarker of the adapted UCLA score denoted as Y, wherein the stress-risk index AUC was 0.77, with a 95% confidence interval (CI) of 0.72, 0.82, for a positive prediction with 85% sensitivity and 48% specificity;
iv) determining a stress-risk index cut-off value by a Youden index that maximizes correct classifications and/or minimizes incorrect classifications and denotes a combination of biomarkers at a determined optimal cut-off value as biomarker V;
v) validating biomarker V by using Y as a dependent variable and V as a predicted probability of positives using a logistic regression model on input continuous biomarkers and confounding risk factors, thereby generating a validated stress-risk index;
vi) discriminating the AUC between the positives and negatives of Y using the predicted probability of positives and using the sensitivity and specificity of correct predictions as diagnostic for predictive validity;
vii) determining an optimal V cut-off value using ROC analysis;
viii) using a non-linear regression model that includes neural networks as comparison to the logistic regression model;
ix) determining the maximum of the Youden index using the ROC curves with the non-linear regression analysis substantiating a functional relationship between the models using multilayer perceptron with two layers and trained with Bayesian regularization, wherein hidden layers have tansig functions and an output layer is linear with ten bootstrap repetitions;
x) optimizing the neural networks and extracting the functional relationship with analysis markers of a stroke risk profile of a patient;
xi) diagnosing a risk of the patient of developing chronic stress and ischemic heart disease related stroke, utilizing the validated stress-risk index for a positive prediction of the stroke risk profile of the patient; and
xii) facilitating a therapeutic decision by authorized personnel for the patient, based on the diagnosis of the risk of developing chronic stress and ischemic heart disease related stroke,
wherein the step xii) of facilitating a therapeutic decision comprises generating, by authorized personnel and based on the diagnosed risk of the patient of developing chronic stress and ischemic heart disease related stroke utilizing the validated stress-risk index, one or more preventative recommendations tailored to the patient, the one or more preventative recommendations being provided to the patient for implementation to reduce the diagnosed risk of the patient of developing chronic stress and ischemic heart disease related stroke.

2. The method according to claim 1, wherein the biological samples obtained are selected from the group consisting of blood, serum, plasma, urine, and saliva.

3. The method according to claim 2, wherein the biological samples obtained are fasting biological samples.

4. The method according to claim 1, wherein the biological samples obtained are fasting biological samples.

5. The method according to claim 1, further comprising:
xiii) treating the patient with the one or more preventative recommendations tailored to the patient.

6. A method for establishing a chronic stress and diabetes related stroke risk phenotype, wherein an adaptation of the UCLA stroke risk score was used to determine the risk of chronic stress and diabetes related stroke in an individual, the method comprising:
i) using the stress-risk index determined according to claim 1 to establish a stress-d-risk index;
ii) performing a statistical analysis of biomarkers and risk factors, wherein variables with skewed non-normal distributions were logarithmically transformed, wherein from among the variables of the adapted UCLA score, the following nine were used as analysis markers: age, sex, systolic blood pressure, use of hypertensive drugs, smoking habit, diabetes, history of cardiovascular disease, electrocardiography (ECG) atrial fibrillation, and electrocardiography left ventricular hypertrophy (ECG-LVH), wherein adaptation of the UCLA stroke risk score included replacement of self-reported values of the analysis markers with quantitative markers including HbA1C≥6.5% as a marker for diabetes and nicotine metabolite cotinine≥14 ng/ml as a marker for smoking, wherein the liver enzyme gamma glutamyl transferase (GGT) was used as a marker for alcohol abuse in developing the stress-d-risk index;
iii) determining standardized values of the analysis markers by principal component analysis (PCA) at baseline;
iv) computing the first principal component scores as a weighted mean of standardized variables with determined weights reflecting seven component loadings that are cotinine, GGT, diabetes defined as HbA1C≥6.5%, systolic blood pressure, perfusion deficits, ECG atrial fibrillation, and ECG-LVH;
v) determining the stress-d-risk index by multiplying the component scores values by ten and increasing it by fifty, such that a mean of the stress-d-risk index is 50 and its standard deviation lies between 0 and minus 100;
vi) determining a cut-point for the stress-d-risk index by conducting a ROC analysis using the cut-off determined in step iv) of claim 1 to discriminate between the positives and negative data and the sensitivity, specificity, and percentage of correct predictions;
vii) denoting a dichotomous variable, which discriminates between those respondents above the cut point and those below the cut point as Y, where the stress-d-risk index AUC was 0.78, with a 95% CI of 0.73, 0.83 for a positive prediction with 81% sensitivity and 59% specificity;
viii) validating the stress-d-risk index by applying multivariate linear regression analysis in a model using logarithmic transformed predictors in a complete dataset of N=349 by using subsets of 10 training sets with each 60% of population and 10 test sets with the remaining 40% of population;
ix) applying a logistic linear regression model by using logarithmic transformed predictors in all data as in step viii) using variable Y as in step vii);
x) validating the logistic linear regression model by repeating step ix) on 10 randomly selected samples and 10 test sets, thereby generating a validated logistic linear regression model;
xi) predicting a probability of risk for chronic stress and diabetes related stroke by obtaining a maximum likelihood estimates of regression coefficients of all regressions;
xii) using the dichotomous variable Y of the stress-d-risk index as in step vii) to discriminate between positives and negatives of the markers of step xi), wherein the stress-d-risk index AUC was 0.82 for a positive prediction;
xiii) using a logistic regression analysis wherein Y is used as the dependent variable and V contains the selected input continuous stress biomarkers as predictors of positives;
xiv) determining an optimal cut-off value for V using ROC analysis;
xv) using the AUC in the ROC analysis on V, using Y and the sensitivity, specificity, and percentage of correct predictions at the cut-off value as diagnostics for a predictive validity of V;
xvi) performing Hosmer-Lemeshow tests for testing goodness of fit for the logistic linear regression model in all participants, training, and test sets;
xvii) diagnosing a risk of the patient of developing chronic stress and diabetes related stroke, utilizing the validated logistic linear regression model; and
xviii) facilitating a therapeutic decision for the patient by authorized personnel, based on the diagnosis of the risk of developing chronic stress and diabetes related stroke,
wherein the step xviii) of facilitating a therapeutic decision comprises generating, by authorized personnel and based on the diagnosed risk of the patient of developing chronic stress and diabetes related stroke utilizing the validated logistic linear regression model, one or more preventative recommendations tailored to the patient, the one or more preventative recommendations being provided to the patient for implementation to reduce the diagnosed risk of the patient of developing chronic stress and diabetes related stroke.

7. The method according to claim 6, wherein the biological samples obtained are selected from the group consisting of blood, serum, plasma, urine, and saliva.

8. The method according to claim 7, wherein the biological samples obtained are fasting biological samples.

9. The method according to claim 6, wherein the biological samples obtained are fasting biological samples.

10. The method according to claim 6, further comprising:
xix) treating the patient with the one or more preventative recommendations tailored to the patient.
